(19) United States
(12) Patent Application Publication
(10) Pub. No.: US 2009/0101764 A1
(43) Pub. Date: Apr. 23, 2009

(54) Device for supporting a medical or non-medical device from an infant warmer

(76) Inventor: Harry A. Tracy, JR., San Antonio, TX (US)

Correspondence Address: Harry A. Tracy, 6103 Royal Point, San Antonio, TX, 78239-1540, US

(21) Appl. No.: 11/975,840

(22) Filed: Oct. 22, 2007

(51) Int. Cl. A61B 19/00

(52) U.S. Cl. 248/59; 248/333; 248/75

(57) ABSTRACT

Prior to this invention, Neonatal Intensive Care Units (NICUs) have used either a piece of umbilical tape, a string of paper clips, or adhesive tape to hold medical or non-medical devices suspended or supported from the upper vertical portion of an infant warmer, incubator, or warmer-incubator combined unit. An example of a medical unit being used in this way is a surgical device called a silo, which is held in a substantially vertical orientation. An example of a non-medical device being supported or suspended from an infant warmer, incubator or warmer-incubator combination unit by the aforementioned umbilical tape, paper clips, or adhesive tape is a mobile. The device claimed in this patent application will replace the use of any locally constructed means of attaching, suspending, or supporting any medical or non-medical device from an infant warmer, incubator, or warmer-incubator combined unit.

### CROSS-REFERENCE TO RELATED APPLICATIONS

Not Applicable

### STATEMENT REGARDING FEDERALLY SPONSORED RESEARCH AND DEVELOPMENT

Development of this device was privately funded.

### REFERENCE TO SEQUENCE LISTING, A TABLE, OR A COMPUTER PROGRAM

### Listing Compact Disk Appendix

Not applicable

### BACKGROUND OF THE INVENTION

1. Field of the Invention

This invention relates to the field of infant care. This invention was developed because there is no means for supporting medical or non-medical devices from an infant warmer, incubator, or warmer-incubator combination unit with a vertically movable canopy. This is important, because there are times when a medical or non-medical device needs to be supported from the upper portion of an infant warmer, incubator, or warmer-incubator combination unit.

An example of a medical device requiring this support system is the use of a silo for the staged reduction of gastroschisis. The silo is a piece of silastic tubing with a spring ring at one end and a loop at the other. Gastroschisis is a congenital birth defect, where a portion of the bowel is exposed to the outside. This condition is due to the abdominal cavity not completely closing during embryonic development. After the infant is born, it is necessary to repair this defect. Unfortunately the exposed viscera, comprising the stomach, small intestines and/or large intestines, takes up more space that the abdominal cavity is capable of holding. In this instance, the viscera must be placed in a protective environment, and allowed to slowly return to the abdominal cavity over several days. The length of the silo is shortened daily, so that more of the viscera are returning to the abdominal cavity by gravity. For this to work properly, the silo must be maintained in a substantially vertical orientation. This is accomplished presently by a piece of umbilical tape, the first end of which is attached to the silo loop, and the second end is attached to the infant warmer, which is above the infant to provide sufficient warmth. Alternatives currently practiced include connecting a string of paper clips together, attaching the first end of the string of paper clips to the silo and the second end of the string of paper clips to the warmer, incubator, or warmer-incubator combination unit. A more recent alternative being used is to place adhesive tape through the silo loop, and attach the tape somewhere above the infant.

There are several different types of infant warmers in use at this time. For example, the new Giraffe OmniBed warmer is an integrated warmer-incubator unit with a vertically movable canopy. This means that there is a plastic cover above the infant, and this cover is lowered to seal off the infant when it is used as an incubator. When used as an infant warmer, the top of the incubator is raised vertically to a position above the infant. This lid has no attachment point from which to hang anything. This unit requires the Neonatal Intensive Care Unit (NICU) personnel to use adhesive tape to hold the silo in a substantially vertical orientation. Another warmer, manufactured by Draeger Medical Inc., has two warmers, which are offset, with a set of lights between the warming units. This warmer also lacks a specific attachment unit for supporting a variety of items, both medical and non-medical. NICU personnel currently must resort to the aforementioned paper clips, umbilical tape, or adhesive tape, to hold a silo in a substantially vertical orientation.

An example of a non-medical device is to support a mobile above the infant. It has been shown that proper development of the infant requires both visual and auditory stimulation. Since many neonates in a NICU are there for many months, it is important that they be provided the stimulation necessary for proper development.

2. Description of Prior Related Art

The prior art that relates to this invention includes previous patents related to infant warmers, incubators, warmer-incubator combination units with vertically movable canopies, heat shrink tubing, mobiles, and support structures. Each component will be addressed separately. Searches were performed on the USPTO Patent Database, and various scientific and scholarly search engines, including, but not limited to, the National Library of Medicine's Medline, PubMed, Google Scholar, Google Patent, PatentStorm.com and freepatentsonline.com.

The patents relating to infant warmers with a vertically movable canopy are:
1. U.S. Pat. No. 7,108,653, “Canopy Adjustable Mounting System for Infant Warming Apparatus”, Sep. 19, 2006, Mackin
2. U.S. Pat. No. 6,953,427, “Infant Care Apparatus with Object Detection Sensing”, Oct. 11, 2005, Mackin, et al.
3. U.S. Pat. No. 6,905,457, “Radiant Field Management for Infant Care”, Jun. 14, 2005, Mackin.
4. U.S. Pat. No. 6,893,390, “Movable Canopy for Infant Apparatus”, May 17, 2005, Mackin.
5. U.S. Pat. No. 6,880,188, “Infant Care Apparatus with Movable Infant Support”, Apr. 19, 2005, Schmidt, et al.
6. U.S. Pat. No. 6,746,394, “Infant Thermal Support Device”, Jun. 8, 2004, Donnelly, et al.
7. U.S. Pat. No. 6,709,384, “Infant Thermal Support Device”, Mar. 23, 2004, Donnelly, et al.
8. U.S. Pat. No. 6,699,173, “Canopy Seal for Infant Care Apparatus”, Mar. 2, 2004, Boris.
9. U.S. Pat. No. 6,669,625, “Movable Canopy for an Infant Care Unit”, Dec. 30, 2003, Costanza, et al.
10. U.S. Pat. No. 6,585,636, “Heater Door Mechanism for Infant Warming Apparatus”, Jul. 1, 2003, Jones, et al.
11. U.S. Pat. No. 6,569,080, “Door Latching Mechanism for Infant Care Apparatus”, May 27, 2003, Dykes, et al.
12. U.S. Pat. No. 6,506,147, “Movable Canopy Warmer for Infant Care Unit”, Jan. 14, 2003, Eustace, et al.
13. U.S. Pat. No. 6,447,442, “Infant Care Apparatus Canopy Force Limiting Device”, Sep. 10, 2002, Richards, et al.
14. U.S. Pat. No. 5,971,914, “Infant Thermal Support Device”, Oct. 26, 1999, Donnelly, et al.

The patents relating to infant warmers without a vertically movable canopy are:
1. U.S. Pat. No. 7,264,586, “Pre-Warm Function for Infant Warmer”, Sep. 4, 2007, Mackin, et al.
2. U.S. Pat. No. 7,008,371, “Patient Thermal Support Devise”, Mar. 7, 2006, Goldberg, et al.
3. U.S. Pat. No. 6,413,205, “Infant Warmer with Light Shield”, Jul. 2, 2002, Finney.
4. U.S. Pat. No. 6,471,634, “Infant Care Apparatus with Bidirectional Sliding Drawer”, Oct. 29, 2002, Dykes, et al.
5. U.S. Pat. No. 5,980,449, “Infant Warmer”, Nov. 9, 1999, Benson, et al.
6. U.S. Pat. No. 5,898,817, “Infant Warmer with Height Adjustment Mechanism”, Apr. 27, 1999, Salmon, et al.
7. U.S. Pat. No. 5,841,944, “Infant Warmer Radiant Heater Head”, Nov. 24, 1998, Hutchinson, et al.
8. U.S. Pat. No. 5,817,002, “Infant Thermal Support Device”, Oct. 6, 1998, Donnelly, et al.
9. U.S. Pat. No. 5,649,896, “Draft Shield for Infant Radiant Warmer”, Jul. 22, 1997, Barsky.
10. U.S. Pat. No. 5,498,229, “Infant Radiant Warmer”, Mar. 12, 1996, Barsky, et al.
11. U.S. Pat. No. 5,453,077, “Infant Thermal Support Device”, Sep. 26, 1995, Donnelly, et al.
12. U.S. Pat. No. 5,162,038, “Infant Warmer Open Bed”, Nov. 10, 1992, Wilker.

The patents relating to incubators are:
1. U.S. Pat. No. 5,242,375, “Infant Incubator and Humidifier”, Sep. 7, 1993, McDonough

The patents relating to heat shrink tubing are:
1. U.S. Pat. No. 5,984,920, “Rotatable Sphincterotome/Papillotome and Method of Use”, Nov. 16, 1999, Steinbach.
2. U.S. Pat. No. 5,606,637, “Decorative Fiber Optic Light Fixture and Methods of Making Same”, Feb. 25, 1997, Dolby.
3. U.S. Pat. No. 5,522,830, “Endoscopic Surgical Instrument”, Jun. 4, 1996, Aranyi.

The patents relating to mobiles are:
1. U.S. Pat. No. 7,202,484, “Infant Stimulator and Environment Sterilizing Device”, Apr. 10, 2007, Tantillo.
2. U.S. Pat. No. 6,964,595, “Child's Infant-Care Play Center”, Nov. 15, 2005, Libretti.
3. U.S. Pat. No. 6,598,837, “Infant Nursing Bottle Holder and Mobile Support”, Jul. 29, 2003, Howard, et al.

The patents relating to support structures are:
1. U.S. Pat. No. 7,090,185, “Device for Supporting a Medical Apparatus”, Aug. 15, 2006, Bryan, et al.
2. U.S. Pat. No. 6,039,293, “Auxiliary Device for Bed-Ridden and Disabled Patients”, Mar. 21, 2000, Minet.
3. U.S. Pat. No. 5,775,654, “Telescoping Cup Holding System”, Jul. 7, 1998, Price.

Inspection of the patents listed under paragraphs [0006], [0007], and [0008], show that infant warmers, incubators, or combination warmer-incubators with a vertically movable canopy have nothing designed to attach anything to the upper section. To date, attachment to infant warmers, incubators and combination warmer-incubator combination units with vertically movable canopies has been accomplished by medical personnel by use of paper clips, umbilical tape, and adhesive tape.

Inspection of patents listed under paragraph [0009] relating to heat shrink tubing, show no use of heat shrink tubing as a means of making a slidable friction device that can have variable force requirements for movement.

Inspection of patents listed in paragraph [0010], relating to the use of mobiles, shows that none of these address connecting or using mobiles in the NICU, where infants are in the aforementioned warmers, incubators and warmer-incubator combination units with vertically movable canopies. It is very important that these infants receive these external stimuli for proper development. At present, NICUs are using adhesive tape, umbilical tape, and paper clips to support mobiles.

Inspection of the patents of paragraph [0011], relating to support devices, shows that these inventions are not designed for use with the aforementioned warmers, incubators, or warmer/incubator combination units with vertically movable canopies.

### BRIEF SUMMARY OF THE INVENTION

This invention provides for a means to attach to infant warmers, incubators, and warmer-incubator combination units with vertically movable canopies. This is obviously a novel and innovative approach to the problem, which someone skilled in the art has heretofore not developed.

This invention makes use of heat shrink tubing to create slidable devices for adjusting the length of the interconnecting unit. The use of heat shrink tubing to create a slidable device, which can be constructed with varying degrees of frictional force has heretofore not been developed. By varying the combination of different diameters and lengths of heat shrink tubing, the frictional force required for movement can be varied. This results in the design of friction devices, which are capable of sufficient friction to maintain and hold objects of variable mass. This lack of use of heat shrink tubing to construct variable friction slide devices points to the novelty of this invention

At present, personnel in the NICU and Special Care Unit (SCU) are also using paper clips, umbilical tape, and adhesive tape to support mobiles, and other devices designed to provide auditory, visual, and/or tactile stimulation to infants. Obviously, no one has been innovative enough prior to this invention to design a device to support audible, visual, and tactile stimulating devices in NICU warmers, incubators, warmer-incubator combination units with vertically movable canopies, and bassinettes. This invention fills this need by providing a means for attaching non-medical devices to an infant warmer, incubator, or warmer-incubator combination unit.

The designers of these infant warmers, incubators, and warmer-incubator combination units with vertically movable canopies did not include an attachment point for the support of medical and non-medical devices. This provides evidence of the novelty, innovativeness, and practicality of this invention, which heretofore no one skilled in the art has thought of designing.

The preferred embodiment of this invention incorporates the novel use of an adjustable length tension rod, originally designed for supporting curtains, as a device to insert inside the canopy of an infant warmer-incubator unit. This provides a means of attaching other devices to the canopy of the infant warmer-incubator. To provide a good connection between the tension rod and the inside of the infant warmer-incubator canopy, the ends of the tension rod are reformed to approximate the shape of the inside of the canopy. The adjusting screw of the tension rod is additionally used to create an attachment assembly for the tension rod, which provides the tension rod with an attachment site for connecting an interconnecting unit to the tension rod. This interconnecting unit will extend from the tension rod inferiorly to provide an additional attachment site for supporting or suspending a medical or non-medical device from the canopy.

An alternative embodiment of the connecting device for use with infant warmers which do not have a canopy, but rather only a warmer unit, is to make use of one or more spring clasps, preferably of the lobster clasp variety, which can be attached to the grill cover of the warming unit of the infant warmer. Any other connecting method devisable by one skilled in the art is considered within the scope and spirit of this disclosure.

The preferred embodiment of the interconnecting unit is a device which comprises one or more spring clasps, preferably lobster clasps, at the first end to connect to the tension rod of the warmer-incubator unit, or alternatively, to the warmer unit, and a spring clasp, preferably a lobster clasp, at the second end to attach to the medical or non-medical device or item. The interconnecting unit is constructed with a set of friction sliders, which allow the health care professional to adjust the length of the interconnecting unit, as required for each application. An alternate embodiment, which could comprise another means devisable by one skilled in the art, is considered within the scope and spirit of this disclosure.

### BRIEF DESCRIPTION OF THE SEVERAL VIEWS OF THE DRAWING

FIG. 1 is the overall appearance of the preferred embodiment of the invention, with each separate unit identified numerically, as viewed inside a vertically movable canopy.

FIG. 2 is the front sectional view of the tension rod.

FIG. 3 is the bottom sectional view of the tension rod.

FIG. 4 is the side sectional view of the tension rod.

FIG. 5 is the front perspective view of the interconnecting unit.

FIG. 6 is the side perspective view of the interconnecting unit.

FIG. 7 is the exploded view of the slidable devices.

### DETAILED DESCRIPTION OF THE INVENTION

This invention provides a means by which health care professionals caring for infants can attach various medical and/or non-medical devices or items from an infant warmer, incubator or warmer-incubator combination unit.

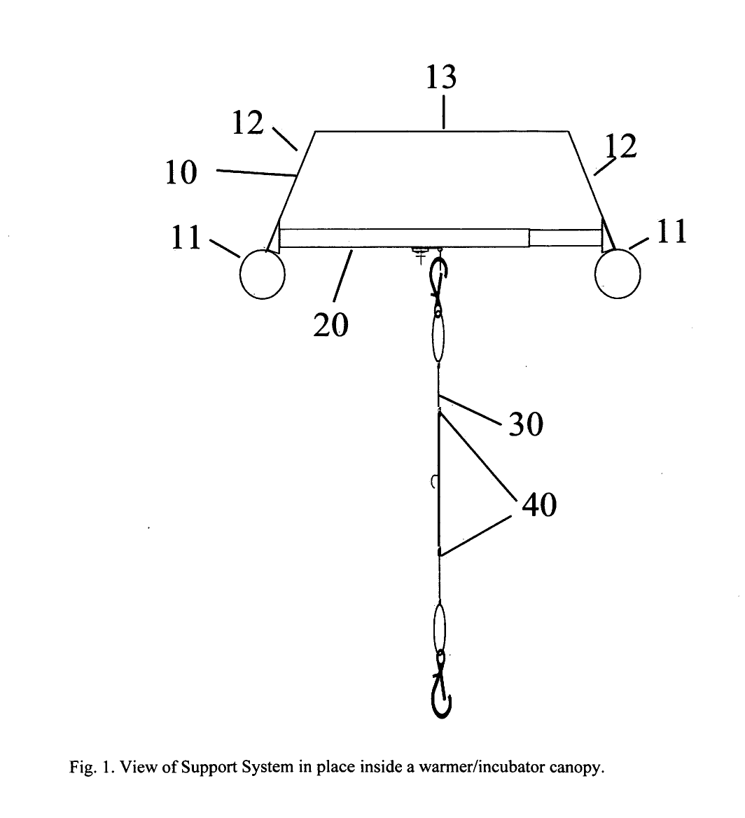

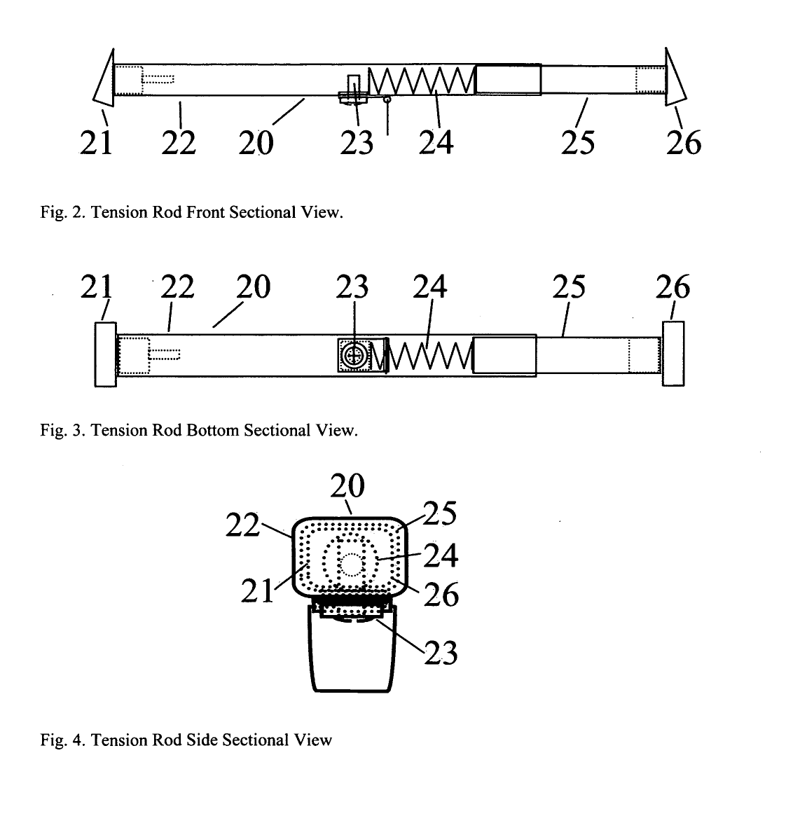

Now turning to the figures in which like references refer to like elements throughout the several views, various aspects and embodiments of the invention are described. The preferred embodiment of the attachment tension rod is constructed from an adjustable tension curtain rod (20), which has two ends with rubber end pieces (21 and 26), and a device (23) for providing an attachment site below the tension rod. In an alternative embodiment, any device with at least one end, which can be maintained inside the canopy (10) that can be designed or constructed by one skilled in the art is considered to fall within the scope and spirit of this disclosure. The construction of the tension rod is as follows, referencing FIG. 1, FIG. 2, FIG. 3, and FIG. 4:
1. The adjustable length tension curtain rod (20) is disassembled by; removing the two end pieces (21 and 26), the attachment assembly (23) by loosening the screw, the slidable portion of the rod (25) and the tension spring (24).
2. The ends of the tension rod (21 and 26) are reformed to approximate the shape of the side wall of the canopy (12), and the seal of the canopy (11). This can be accomplished by use of a saw, or a hot knife, although any other method devisabled by one skilled in the art is considered to fall within the scope and spirit of this disclosure.
3. The end piece (21) for the stable or first end of the tension rod (22) is reattached.
4. The desired midpoint for the unit is measured from the end of the stable, or larger section (22) of the tension rod (20). For the preferred embodiment, this is 10.75 inches from the end of the stable section (22) of the tension rod (20), including the end piece (21).
5. The attachment assembly (23) comprising the screw removed in step one, an attachment loop, which in the preferred embodiment is a mirror hanger, and the nut that goes inside the tension rod, which was also removed in step 1 are put together, but not tightened.
6. The attachment assembly (23) is inserted into the larger section (22) of the tension rod, and positioned so that the attachment loop of the attachment assembly (23) is at the measured midpoint.
7. The screw of the attachment assembly (23) is tightened so that the attachment loop is fixed at the measured midpoint. In an alternate embodiment, this attachment assembly could be any design, fixed or movable, by any means devisable by one skilled in the art, and this design is considered to fall within the scope and spirit of this disclosure.
8. The tension spring (24) is reinserted into the stable section (22) of the tension rod (20).
9. The movable section (25) of the tension rod is inserted into the stable section (22).
10. The appropriate length of the total tension rod (20) is measured from the stable first end of the tension rod (20) with the end piece (21) in place to the proper length on the smaller movable section (25) of the tension rod, minus the length of the end piece (26) of the second end of the tension rod. In the preferred embodiment, this length is 21.5 inches, measured from first end piece (21) to second end piece (26)
11. The smaller, movable section (25) of the tension rod is then cut to the length measured in step 10.
12. The end piece of the second end (26) is then inserted into the end of the small movable section (25) of the tension rod (20).

To attach the tension rod to the infant warmer-incubator combination unit, the health care professional will insert the larger end (22) into the vertically movable canopy, such that the larger end (22) is positioned above the seal (11) and below the top section (13), and the larger end piece (21) is pressed against the side section (12) and the seal (11). The smaller end (25) of the tension rod is then compressed and placed inside the canopy, such that the small end piece (26) is positioned above the seal (11) and below the top section (13) and the smaller end piece (26) is pressed against the side section (12) and the seal (11) when the compression force is released.

Alternate embodiments of the attachment tension rod include, but are not limited to, a different design for the warmer-incubator combination unit, or the use of one or more spring clamps for use with other infant warmers or incubators, to be connected to the cover grill of the radiant heater or heaters. Said alternate embodiments can be of any design devisable by one skilled in the art, and are considered to be within the scope and spirit of this disclosure.

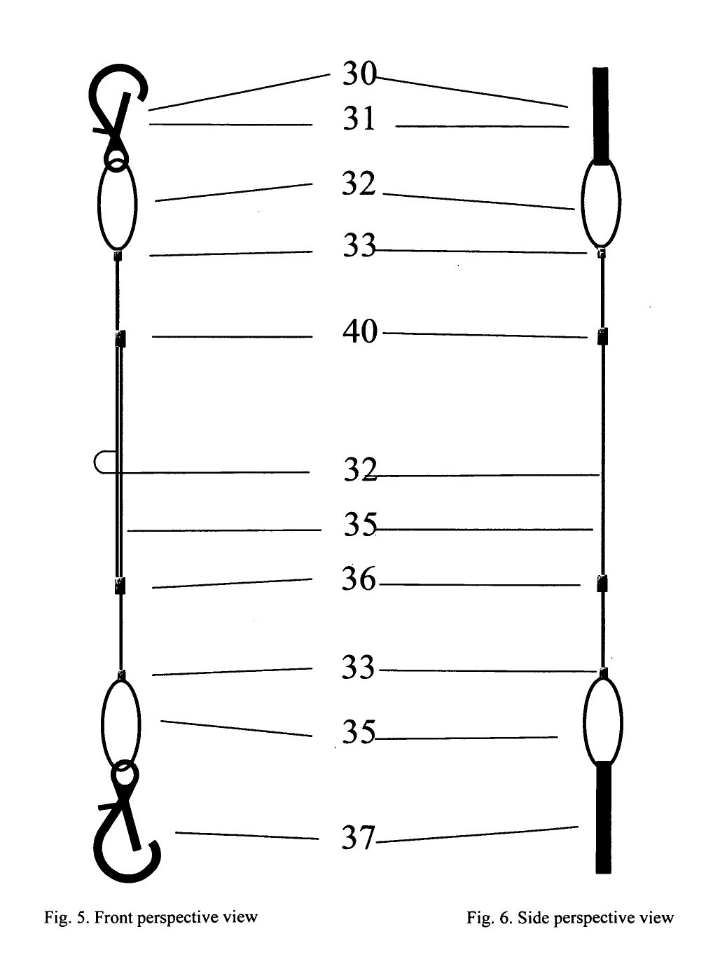

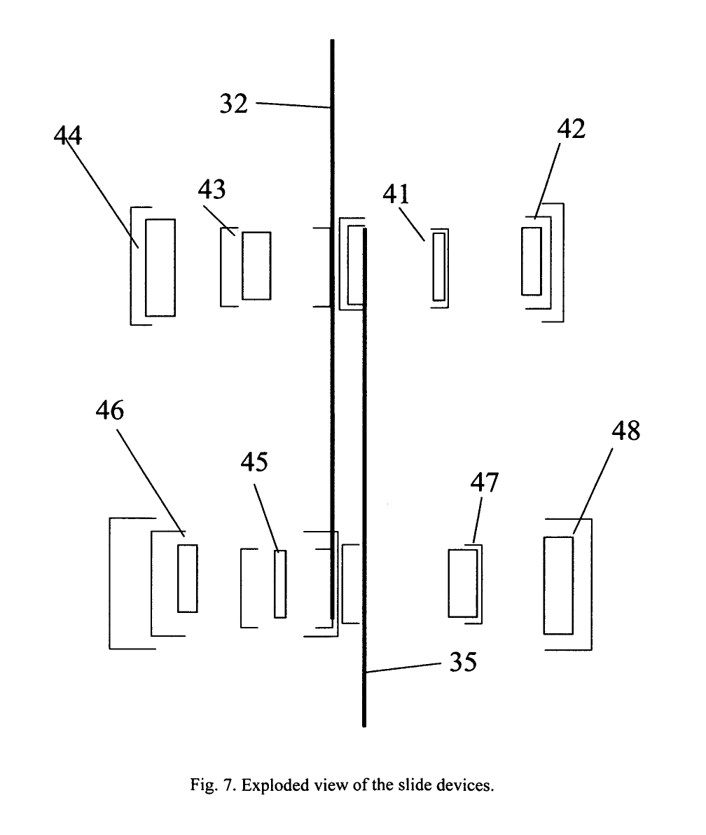

The preferred embodiment of the interconnecting unit is constructed from 1/16 inch steel rope, heat shrink tubing in various diameters, spring clasps preferably of the lobster variety, and crimp connectors, though any alternate embodiment of this unit devisable by one skilled in the art is considered to be within the scope and spirit of this disclosure. The following steps, relating to FIGS. 5, 6, and 7, complete the construction of the interconnecting unit:
1. The steel cable is cut to the appropriate length. For the preferred embodiment, this length is 20 inches for the upper steel rope section (32) and 8 inches for the bottom steel rope section (35).
2. One end of each piece of steel rope (32 and 35) is bent one inch from the end, and this bend will be used to form a loop through the spring clasps (31 and 37).
3. The upper steel rope section (32) will have the bent end inserted through the loop of the upper spring clasp (31), which is a 29 mm spring clasp of the lobster variety in the preferred embodiment. An alternate embodiment made from any connecting device that can be devised by one skilled in the art will be considered within the scope and spirit of this disclosure.
4. The bent end of the upper steel rope (32) is folded back on itself, to form a 1-inch loop through the upper spring clasp (31). The point where the bent end of the steel rope touches back on itself is then connected together using a crimp connector (33). After crimping the junction, it is sealed using solder. An alternate embodiment made from any connecting device that can be devised by one skilled in the art will be considered within the scope and spirit of this disclosure.
5. The lower steel rope section (35) will have the bent end inserted through the loop of the lower spring clasp (37), which is a 31 mm spring clasp of the lobster variety in the preferred embodiment. An alternate embodiment made from any connecting device that can be devised by one skilled in the art will be considered within the scope and spirit of this disclosure.
6. The bent end of the lower steel rope (35) is folded back on itself, to form a 1-inch loop through the lower spring clasp (37). The point where the bent end of the steel rope touches back on itself is connected together using a crimp connector (33). After crimping the junction, it is sealed using solder. An alternate embodiment made from any connecting device that can be devised by one skilled in the art will be considered within the scope and spirit of this disclosure.
7. This completes the construction of the upper and lower portions of the interconnecting unit.
8. The next step is to join the two sections of steel rope (32 and 35), with the Slide Devices (40 and 36).

Alternate embodiments of the interconnecting unit include, but are not limited to, the use of alternate materials or designs. Said alternate embodiments can be of any design devisable by one skilled in the art, and are considered to be within the scope and spirit of this disclosure.

The preferred embodiment of the slide device comprises various lengths of heat shrink tubing, assembled so that the two sections of steel rope can be adjusted relative to each other, though any sliding device devisable by one skilled in the art is considered to fall within the scope and spirit of this disclosure. The slide devices are constructed as follows:
1. The unbent end of each section of steel rope (32 and 35) will have a ½ inch long piece of 1/16 inch diameter heat shrink tubing (41 and 45) placed over the end. These ends are then subjected to a heat source, such as a hot air gun, until each piece of heat shrink tubing (41 and 45) has shrunk tightly around the end of each section of steel rope (32 and 35). Any alternate heat source can be used and is considered to fall within the scope and spirit of this disclosure.
2. Two ½ inch long piece of ⅛-inch diameter heat shrink tubing (42 and 46) is then placed over the previously shrunk pieces of tubing (41 and 45). These ends are then subjected to a heat source, such as a hot air gun, until each piece of heat shrink tubing (42 and 46) has shrunk tightly around the previously shrunk tubing (41 and 45). Any alternate heat source can be used and is considered to fall within the scope and spirit of this disclosure.
3. Two ½ inch pieces of ⅜ inch diameter heat shrink tubing (43 and 47) are then placed over each piece of steel rope (32 and 35), separate from the previously shrunk sections of heat shrink tubing (41, 42, 45, and 46). These two pieces are then subjected to a heat source, such as a hot air gun, until each piece of heat shrink tubing (43 and 47) has shrunk. These sections are not tight around the steel ropes (32 and 35). Any alternate heat source can be used and is considered to fall within the scope and spirit of this disclosure.
4. The two sections of steel rope (32 and 35) are then aligned in opposite directions such that the previously constructed sets of heat shrunk tubing (41, 42, 45, and 46) are opposite to the two pieces of heat shrink tubing constructed loosely in step 11 (43 and 47).
5. Each of these opposed sections are then covered with a ¾ inch long section of ⅜ inch diameter heat shrink tubing (44 and 48). These pieces of heat shrink tubing (44 and 48) are then subjected to a heat source, such as a hot air gun, until each piece of heat shrink tubing (44 and 48) have shrunk tightly around the previously shrunk tubing (41, 42, 43, 45, 46, 47, respectively). This results in a set of slide mechanisms that can be adjusted easily to set the length of the interconnecting unit to the proper dimension.

Alternate embodiments of the slide device include, but are not limited to, the use of alternate materials or designs. Said alternate embodiments can be of any design devisable by one skilled in the art, and are considered to be within the scope and spirit of this disclosure.

To use the device, a health-care professional begins by placing the first end of the tension rod inside the canopy of the infant warmer-incubator combined unit, compressing the second end of the tension rod, and positioning the second end opposite the first end inside the canopy. The interconnecting unit is then attached by clipping the lower spring clasp (37) of the interconnecting unit to the medical or non-medical device to be supported or suspended from the upper portion of the infant warmer-incubator combination unit (not shown). The length of the support is adjusted by pulling the upper spring clasp (31) and the lower spring clasp (37), away from each other. Once the appropriate length is established, the upper spring clasp (31) is attached to the tension rod (20) previously placed within the canopy. The length can then be adjusted longer by pulling the upper spring clasp (31) and the lower spring clasp (37) away from each other, or shorter by pulling the upper slide device (40) and the lower slide device (36) away from each other. Once the appropriate length is achieved, no further adjustment is required, unless the health-care professional deems it necessary.

What is claimed:

1. An apparatus for attaching medical or non-medical devices to an infant warmer, incubator, or combination thereof, the apparatus comprising
a. At least one component;
b. At least one end;
c. A means to attach to an infant warmer, incubator, or warmer-incubator combination unit;
d. The said attachment can be either permanent or temporary;
e. The attachment can be an accessory designed after the infant warmer, incubator, or combination thereof has been designed or manufactured;
f. The attachment can be designed into future infant warmers, incubators, or combinations thereof;
g. Said attachment means can be by any method devisable by one skilled in the art.
h. An interconnecting device;
i. A means for attaching a medical or non-medical device or item to the interconnecting device;
j. Can be composed of any material devised by one skilled in the art; and
k. Any alternate embodiment of this apparatus is considered within the scope and spirit of this disclosure.

2. The invention recited in claim 1 has at least one attachment point

3. The invention recited in claim 1 can provide for the attachment of an interconnecting unit:
a. Said attachment may be either fixed or removable; and
b. Said attachment can be by any means devisable by one skilled in the art.

4. The interconnecting unit comprising:
a. At least one component;
b. Has at least one end;
c. A means for connecting to the attachment unit;
d. Is composed of any material devisable by one skilled in the art;
e. Can be fixed or movable;
f. Can be permanent or temporary;
g. Can be of fixed or variable length;
h. Provides at least one attachment point;
i. Said attachment can be by any means devisable by one skilled in the art; and
j. The length of the interconnecting device can be adjusted by means of a slidable device; and
k. The apparatus can be used for medical or non-medical devices;.

5. The apparatus recited in claim 4 is capable of attaching, supporting, or suspending:
a. A medical or non-medical device or item that can be attached to the interconnecting unit by any method devisable by one skilled in the art;
b. Said medical device or item can include, but is not limited to, a gastroschisis staged reduction silo, an oxygen hose, a ventilator tube, or a feeding tube;
c. Said non-medical device or item can include, but is not limited to, a stimulating device, or a monitoring device;
d. Said stimulating device or item can include, but is not limited to, auditory, visual, or tactile stimulation.

6. The apparatus recited in claim 4 comprising:
a. A slide device whereby the length of the unit can be varied;
b. At least one slide device;
c. The slide device is capable of supporting or suspending any medical or non-medical device or item; and
d. The slide device is any device that can be devised by one skilled in the art.

7. A slide device comprising:
a. Various sections of heat shrink tubing.

8. The slide device recited in claim 7, comprising:
a. Various diameters and lengths of heat shrink tubing;
b. Combinations of various diameters and lengths of heat shrink tubing will determine the frictional force of the slide device;
c. A piece of material can be inserted inside the heat shrink tubing to provide additional control of the amount of frictional force developed by the slide device; and
d. Said heat shrink tubing and material can alternatively be any material devisable by one skilled in the art.
